(19) United States
(12) Patent Application Publication
(10) Pub. No.: US 2010/0152538 A1
(43) Pub. Date: Jun. 17, 2010

(54) DEVICE FOR PREVENTING ENDOSCOPE DAMAGE BY ERRANT LASER FIRE IN A SURGICAL LASER

(76) Inventors: Christopher M. Gleason, Tucson, AZ (US); Myles J. Stone, Tuscon, AZ (US)

Correspondence Address: PERRY E. VAN OVER & ASSOCIATES, PLLC, P.O. BOX 399, FAIRFAX, VA, 22038, US

(21) Appl. No.: 12/337,620

(22) Filed: Dec. 17, 2008

(51) Int. Cl. A61B 1/00

(52) U.S. Cl. 600/117

(57) ABSTRACT

Provided is a device that can be attached to an endoscopic device and when so attached is capable of protecting said endoscope from damage that may be caused by errant laser fire. Also provided is a device that can determine the potential for damage from errant laser fire to a connected endoscopic device and emit a warning of such potential damage and, if required, can place the laser in a standby mode to avoid damage from errant laser fire.

### BACKGROUND OF THE INVENTION

1. Technical Field

The present invention relates to endoscopic devices and particularly to endoscopic devices with an errant laser fire damage avoidance device component. More particularly the present invention relates to a laser damage prevention component that can be incorporated into an endoscope, the damage avoidance device being capable of alerting the user when laser emissions are close enough to cause damage to the fragile endoscopic equipment and further being capable of putting the laser into a standby mode before damage to the endoscope can occur.

2. Background Art

The use of laser radiation has become an accepted medical technique in many areas of modern diagnostic and surgical medical practice. High energy lasers can affect considerable damage to fragile endoscopic equipment if the laser fiber is withdrawn by the user too close into the endoscope so as to damage the metal, plastic, glass and/or glue used in the structure of the endoscope. Such damage frequently occurs because the surgeon is focused upon the application of laser energy to the affected tissues and inadvertently draws the active laser emitting fiber into the instrument, resulting in the device being put out of service and requiring expensive repairs. Further, such damage to the device can affect patient safety if a resulting metal shard or glass fragment becomes embedded in the tissue of the patient or un-aimed or errant laser energy (from flexible scope) inadvertently strikes a patient's tissue.

Some conventional endoscopic devices such as those described in U.S. Pat. No. 5,222,953; U.S. Pat. No. 6,135,996; U.S. Pat. No. 6,464,693; U.S. Pat. No. 6,544,257; and published U.S. Patent Application No. 2004/0199151 have integrally provided laser fiber movement controlling devices that depend upon the use of heat sensors, fiber advance and retracting speed limiting mechanisms, magnetic position sensors, light sensitive monitoring elements or the like in an effort to provide some degree of protective control over the movement of the laser fiber. While each of these exemplary conventional endoscopes employ widely different means to provide some control and limited protection for the use of the high intensity laser components, they each use a generally common endoscopic and laser transmitting technology as is well known in the art. The conventional and well known basic components of each of these referenced endoscopes and laser emitters are fully incorporated herein by reference. As discussed above, these referenced devices and some other such conventional endoscopic instruments also include integrally manufactured and widely varied laser fiber movement controlling components, each of which, for different reasons suffers from a conceptual limitation resulting in limited and ineffective protection for the devices from errant laser fire. Further, none of the movement limiting efforts of these conventional devices is configured as a separate device that can be easily adapted and attached to any conventional endoscope and when so attached can provide effective protection from errant laser fire.

There is therefore, an unfulfilled need for an effective, dependable protective device component that can be adapted to and attached to any existing endoscope so as to provide a warning to the surgeon-user of potential damage that may occur to the structure of the endoscope when the laser emitting fiber optic element is withdrawn into the endoscope, and further provides a protective device that is capable of setting the laser into a standby mode before damage can occur to the endoscope.

### SUMMARY OF THE DISCLOSURE

The present invention meets the above identified need by providing an easily attachable endoscope saving device that can be adapted for attachment to any conventionally produced endoscope in place of, or attached to, a standard fiber port seal, wherein the endoscope saving device is capable of initiating a warning signal to the user when the laser fiber is being withdrawn so close into the endoscope so as to constitute a potential danger from errant laser fire and if the laser fiber is withdrawn into the endoscope will automatically cause the laser to be put into a standby mode.

Also provided is an endoscope saving device that can automatically calculate how far the laser fiber extends past the port seal of the device, the device being easily adapted to any conventional endoscope by the users pre-selecting the type of endoscope to be used and attaching the device to the endoscope in place of a standard fiber port seal.

Also provided is an endoscope saving device capable of monitoring the amount of fiber inserted into the scope and comparing it to a stored database of standard or custom scope lengths.

Also provided is an endoscope saving device that includes a roller unit and a functionally connected data processor, the roller unit being capable of attachment to any conventional endoscope in place of, or attached to, a standard fiber port seal and the data processor being programmable to include a stored database of standard or custom scope lengths.

Also provided is an endoscope saving device having a roller unit component that is easily adapted to and attachable to any conventional endoscope, the roller unit being small, lightweight, and waterproof so as to be capable of chemical and/or heat sterilization.

Also provided is an endoscope saving device having a data processor operationally connected to, or integrated into, a roller unit, the roller unit being biased so as to contact a laser fiber passing through an endoscope, the roller unit being capable of tracking movement of the fiber in contact therewith and transmitting that movement data to the data processor such that when preset parameters are met, the processor, by calculating the amount of fiber inserted into the scope and comparing it to a stored database of standard or custom scope lengths, is capable of determining a potential errant laser fire threat to the endoscope structure and initiating a warning to the user and, if the parameters of the warning are exceeded to set the laser in a standby mode by triggering the standard laser safety interlock.

Also provided is an endoscope saving device having a control interface capable of permitting data input and entering of command instructions such as menu selection and standby mode selection to the processor of the device and displaying and saving input data as well as a record of data collected by and transmitted from the roller unit of the device.

Also provided is an endoscopic instrument in combination with the endoscope saving device of the present invention, the combination being assembled as two separate manufactures or being provided as a unified manufactured device.

Also provided is a method of using an endoscope for medical diagnostic or surgical procedures wherein a step in the method includes providing the endoscope saving device of the present invention and adapting that device to a selected endoscope, entering data relative to the selected endoscope into the data processor of the device through the control interface, and operating the endoscope and laser unit so as to effect a diagnostic or surgical procedure wherein the endoscope is protected from errant laser fire.

Also provided is a kit, which includes at least one endoscope saving device provided in accordance with the present invention.

### BRIEF DESCRIPTION OF THE DRAWINGS

The foregoing and other features of the novel endoscope saving device will become apparent to one skilled in the art to which the disclosed system and devices relate upon consideration of the following description of exemplary embodiments with reference to the accompanying drawings, wherein:

FIG. 1 shows the endoscope saving device of the present invention having an opposing dual roller component and configured for attachment to an endoscope in place of a standard fiber port seal.

FIG. 2 shows a diagrammatic representation of the endoscope saving device having a roller unit and a data processor with a control interface in operational combination with an endoscope and a laser controlling unit.

FIG. 3 shows an embodiment of the present invention wherein tracking of the fiber movement is accomplished using an optical roller having reflective markings on the roller and the tracking data acquired by a sealed optical sensor and transmitted to a data processor by electrically conductive wire.

FIG. 4 shows an embodiment of the present invention wherein tracking of the fiber movement is accomplished using an optical roller having reflective markings on the roller and the tracking data acquired by and transferred by an optical fiber connected to an optical sensor associated with the data processor.

FIG. 5 shows an embodiment of the present invention wherein tracking of the fiber movement is accomplished as relative movement of graduated indicia on the fiber are read by an optical sensor connected to the data processor by electrically conductive wire.

FIG. 6 shows an embodiment of the present invention wherein tracking of the fiber movement is accomplished as movement of the fiber causes an attached two-stage label to pass by an optical sensor connected to the data processor by electrically conductive wire.

FIG. 7 shows an embodiment of the present invention wherein tracking of the fiber movement is accomplished as relative movement of the fiber is tracked and transmitted by an optical fiber connection to an optical sensor, which in combination with the data processor determines the degree of fiber movement.

FIG. 8 shows an embodiment of the present invention wherein tracking of the fiber movement is accomplished as relative movement of the fiber is tracked by an optical sensor, the tracking data being transmitted to the data processor by electrically conductive wire.

FIG. 9 shows an embodiment of the present invention wherein tracking of the fiber movement is accomplished as a mechanical switch is contacted and physically moved by a switch contact element attached to a pre-selected position on the fiber as the fiber passes the mechanical switch. Movement of the mechanical switch serves to close or open a circuit that sends a signal to the data processor indicating the position of the fiber.

FIG. 10 shows an embodiment of the present invention wherein tracking of the fiber movement is accomplished as a magnetic/capacitive tracking unit that is affixed to a pre-selected position on the fiber distal to a tracking sensor moves to a measured distance position near the tracking sensor, the signal from which is transmitted to the data processor by an electrically conductive wire.

FIG. 11 shows an embodiment of the present invention wherein tracking of the fiber movement is accomplished as a magnetic/capacitive tracking unit that is affixed to a pre-selected position on the fiber proximal to a tracking sensor, such as in the handle control of the laser, moves to a measured distance position near the tracking sensor, the signal from which is transmitted to the data processor by an electrically conductive wire.

FIG. 12 shows an embodiment of the present invention wherein tracking of the fiber movement is accomplished as a magnetic/capacitive tracking unit that is affixed onto the distal tip of the fiber is drawn sufficiently close to a proximity sensor located at the distal end of the endoscope structure such that a signal indicating close proximity of the tracking unit to the proximity sensor is sent by an electrically conductive wire to the data processor.

FIG. 13 shows an embodiment of the present invention wherein tracking of the fiber movement is accomplished by the heat generating distal end of the fiber comes into proximity to a heat sensor affixed to the distal end of the endoscope structure such that a signal is generated by the heat sensor and transmitted to the data processor by an electrically conductive wire.

### DETAILED DESCRIPTION OF PREFERRED EMBODIMENTS

Detailed embodiments of the present invention are disclosed herein; however, it is understood that the following description and each of the accompanying figures are provided as being exemplary of the invention, which may be embodied in various forms without departing from the scope of the claimed invention. Thus, the specific structural and functional details provided in the following description are non-limiting, but serve merely as a basis for the invention as defined by the claims provided herewith.

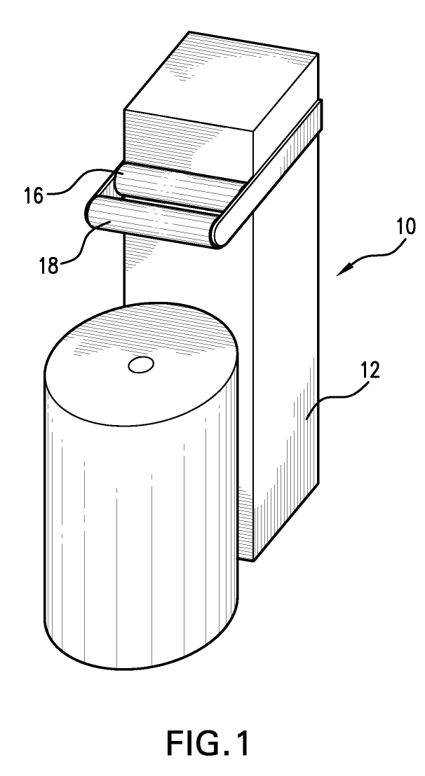

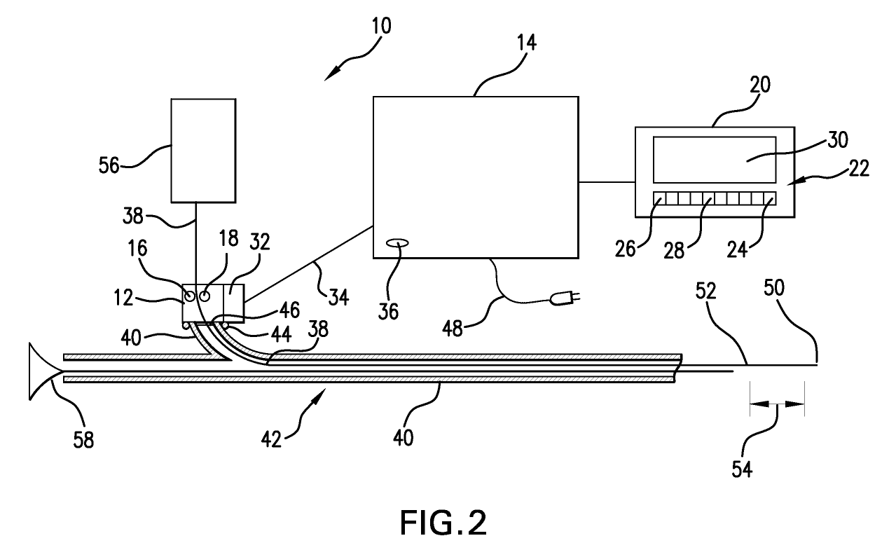

The endoscope saving device, as generally depicted at 10 in the accompanying figures, includes a roller unit 12 as best shown in FIG. 1, and a data processor 14 as diagrammatically presented in FIG. 2. The endoscope saving device 10, when used in combination with any endoscope, such as, for example those described in the above reference and fully incorporated patents and the published patent application can provide effective protection of the endoscope structure from errant laser fire by warning the user of proximity of the distal end of the fiber 38 to the endoscope and if the tip is withdrawn into the endoscope to automatically set the laser to standby.

As shown in FIG. 1, a preferred embodiment of the present invention includes a roller unit 12 having two opposing marked rollers 16, 18, which are sufficiently biased one toward the other so as to force sufficient contact with the fiber 38, which will traverse between the marked rollers 16, 18 and by doing so accurately transmit the degree of fiber 38 movement. Movement of the marked rollers 16, 18 and thus movement of the fiber 38 passing between the rollers 16, 18 is tracked by an optical sensor 32, best displayed graphically in FIG. 2. The movement data collected by the optical sensor 32 is transmitted by a data transmission connector 34 to the processor 14 for analysis, display, storage, and when required downloading via at least one processor data port 36. The transmission connector 34 between the roller unit 12 and the processor 14 can be an electrically connective wire or fiber optic cable. Importantly, the roller unit 12 and the transmission connector 34 can both be configured so as to be easily removed from the endoscope to permit sterilization in preparation for subsequent reuse with other endoscopes. The roller unit 12 preferably is small and light weight and substantially waterproof so as to tolerate chemical and/or heat sterilization.

The roller unit 12 can be configured so as to be capable of attachment to all conventionally known endoscopes. If applicable, the device can be attached to the inner sheath as a modified replacement for, or attachment to a standard fiber port seal, which is required on all endoscopes currently in production. Because the fitting is an industry wide standard, the endoscope saving device 10 is sized and configured so as to be adaptable to fit all known endoscopes.

Similar to the required standard fiber port seal, the roller unit 12 of the endoscope saving device 10 serves to prevent leakage around the fiber 38 and in addition contains a sealed, sterilizable roller unit for accurately measuring how far the fiber 38, which passes between the two opposing marked rollers 16, 18 has been inserted in the endoscopic device.

The endoscope saving device 10 automatically calculates how far the laser fiber 38 extends past the distal end of the endoscope by monitoring or tracking the amount of fiber 38 inserted into the scope and comparing it to a stored database of standard or custom scope lengths. Prior to use, the surgeon selects which scope is to be used and enters that information into the data processor using the control interface 20. The control interface 20 includes a data input keyboard 22 that can include a standby selector 24 that serves to permit routine removals, a menu selector 26 that permits adjustment of settings, and a data entry touchpad 28 that enables a user to directly input data, select options, and generally interface with the software of the processor 14. A display screen 30 can be provided to facilitate exchange of information from the processor to the user. The processor data port 36 permits high speed data uploads and/or downloads as needed. The processor 14 can be the input point for a power source 48 that can be transferred through the data transmission connector 34 to the roller unit 12. The power supply for the endoscope saving device 10 can be through an inline AC/DC transformer connected to a hospital grade plug or by battery power as the operational needs dictate.

As graphically displayed in FIG. 2, the roller unit 12 can be attached to the laser fiber port 40 of the endoscope, generally shown at 42. It is a particular advantage of the present invention that the connection of the roller unit to the endoscope is by connection at the standard fiber port, which is standardized for all currently manufactured endoscopes. This standardization makes it possible for the endoscope saving device 10 to be easily adapted to and attached to any conventional endoscope as compared with other conventional fiber protection control devices that are particularly designed and manufactured to fit a only a specific endoscope make and model. The roller unit 12 of the present invention can have a flexible rubber gasket that can surround the flanges 46 and create a seal around the sheath 40 thus providing a substantially waterproof connection.

In addition to the inward bias of the opposing rollers 16, 18, the contact of the rollers 16, 18 with the fiber 38 that passes between them can be facilitated by manufacturing the rollers 16, 18 of a material having a suitable texture or material so as to have a coefficient of friction in contact with the fiber 38 so as to result in an accurate and true tracking of the fiber 38 movement without impeding that movement. In an alternative configuration, the entire roller unit 12 can rotate with the fiber around the sheaths fiber port, which could permit tracking of rotational movement and thus help quantify proper lasing technique.

Medical lasers all require an interlock circuit. If this circuit is broken the laser will be placed into a standby mode. The circuit contains two external probes, connected by a small jumper cable housed in a plastic or metal plug. Typically, the circuit power varies by manufacturer, but generally will not exceed a few milliamps operating across 12 volts. In practice, the endoscope saving device 10 will connect the jumpers during normal operation, but will break the circuit if the laser fiber is retracted too far within the structure of the endoscope. The interlock plugs are proprietary connections formed of plastic and/or metal. Each manufacturer tends to use a unique plug shape although each has the same function. The endoscope saving device can be provided in one of three configurations: preinstalled with a replicated plug attached, as a bare cable with instructions outlining how users can retrofit their own plugs, or with the users' provided plug attached prior to shipping the purchasers order for the endoscope saving device. Other options can also be conceived by which the problem of the current state of using proprietary plugs can be addressed without departing from the spirit of the invention.

Essential to the operation of the endoscope saving device 10 is the provision of appropriate software to be designed for and integrated into the data processor 14. While the software is an essential part of the operation of the device 10, it is not beyond the easy capability of those currently skilled in the art to provide and upload the software into the endoscope saving device 10. The software can be designed to calculate how far the fiber 38 extends past the distal end of the endoscope. The software will be capable of producing a warning tone through the data processor 14 when the fiber approaches too close to the distal end of the endoscope. As shown graphically in FIG. 2, between the distal end of the laser fiber 38 (aka 50?) and the laser shut-off point (52?) is a warning zone 54. It is within this warning zone 54 that the software can be designed to emit the warning tone. If the user through manipulation of the laser fiber 38 causes the distal end of the laser fiber 50 to get closer to the endoscope structure than that point programmed as the warning zone 54, the endoscope saving device 10 will break the interlock circuit of the laser, switching it into a standby mode, thus saving the endoscope 42 from damage by errant laser fire. As depicted in FIG. 2 at 52, the endoscope lens is located at the most distal point of the endoscope 42 and while adequate for viewing the tissue of interest in the patient, it is of little practical use in monitoring the potential danger that the laser fire represents to the structure of the endoscope. With the assistance of the endoscope saving device 10 can such damage be avoided.

In addition to the primary functions of warning the user of laser fire proximity to the endoscope and, if necessary, putting the laser in a standby mode to protect the endoscope from damage, the software for the device 10 can include storing standard scope lengths and allowing for the programming of custom lengths when necessary. The device 10 can also be used in manual mode wherein a simple activator, such as, for example a button located anywhere on the device 10 or the “enter” key on the key pad 22 of the data processor can be used to set the shut-off point when the fiber is at the point of causing damage. The warning zone 54 can then be calculated automatically by adding a factory pre-determined value onto the amount of fiber inserted when the distance was manually set.

If the standby selector 24 is pressed by the user during normal operation, the endoscope saving device 10 will allow the fiber 38 to be removed without a warning tone or tripping the laser interlock and initiating a standby mode. Additionally, the device 10 can display whether the device 10 is in standby or active mode via the display screen 30 or, alternatively, a status light.

Further, the device 10 can be programmed to log the date and time of all warning tones and standby modes. These can be referenced to verify that the unit was in use, as well as to retain a record of the parameters of such use. Usage data can also be stored so as to track the rate of fiber 38 entry and withdrawal and potentially fiber rotation if that optional capability is needed. This will help quantify the science of laser surgery by correlating fiber 38 movement with positive patient outcomes. Basic usage data, such as warning tone and standby mode history, cumulative use, number of removals and such can be stored and later made available for display on the screen 30. Voluminous data can be downloaded from the processor 14 through the data port 36. Programming can be designed to permit logging of events across the entire lifetime of each individual device 10 as well as for the length of each case.

Additional programming can be provided to retain, analyze, and/or display events of normal operations of time, date, standby mode, active mode, make and model of endoscope that was selected, running length of the fiber 38 inserted to very low tolerances such as a tenth of a millimeter or less, the target values for warning zone and standby mode initiation. If applicable, remaining battery life can also be provided as a standard display. The capacity of the processor 14 can be provided so as to enable virtually any combination of displays and analysis as dictated by the software selection for the device 10.

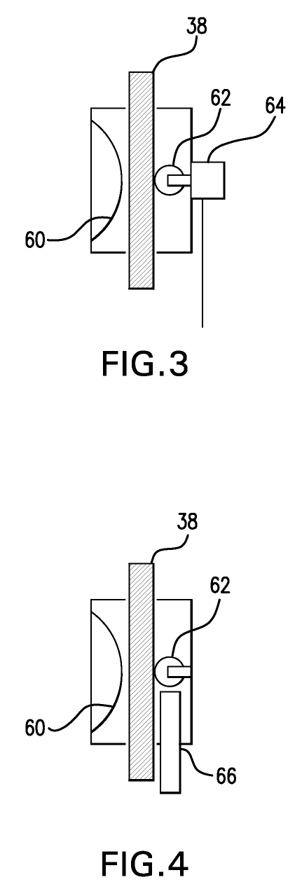

In addition to the preferred embodiment of the endoscope saving device 10 described above, it is within the general principles of the invention that alternative embodiments as shown in FIGS. 3-13 and described below can also be provided.

As shown in FIG. 3, the roller unit 12 of the endoscope saving device 10 can be configured to track passage of the fiber 38 between a biasing member 60, such as a spring pad, for example, and a single marked roller 62 having indicia such as reflective markings. In such an embodiment, tracking of the roller 62 movement and therefore the movement of the fiber 38 could be determined by monitoring the movement of the roller indicia by a sealed optical sensor 64. The alternative embodiment shown in FIG. 4 is similar to that of FIG. 3 with the difference that instead of transmitting the data by electrically conductive wire from an optical sensor, the indicia of the single roller 62 provides tracking data to the processor 14 through an optical fiber 66.

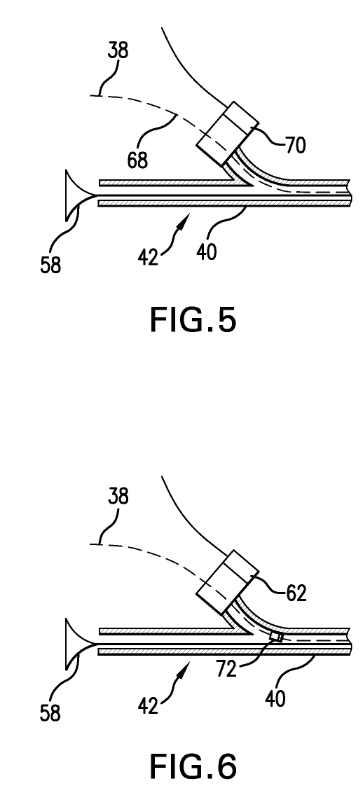

As shown in FIG. 5, an alternative embodiment of the device 10 can be provided wherein the fiber 38 is provided with graduated indicia or markings 68, which are tracked by an optical counter 70 with the data being transferred to the processor 14 through an electrically conductive wire. The indicia can be analog or digital encoding on the laser fiber.

Similar to the alternative device shown in FIG. 5, the alternative roller unit 12 shown in FIG. 6 tracks movement of the fiber 38 by providing position indicia on the fiber 38 itself. In this alternative embodiment, the position indicia is provided as a two-state label 72 as exemplified by two distinct colors, one color for a warning and the second color to initiate standby mode for the device 10. The tracking of movement of these two color indicia and thus the movement of the fiber 38 is by an optical sensor 62 with the data being transferred to the processor 14 by an electrically conductive wire.

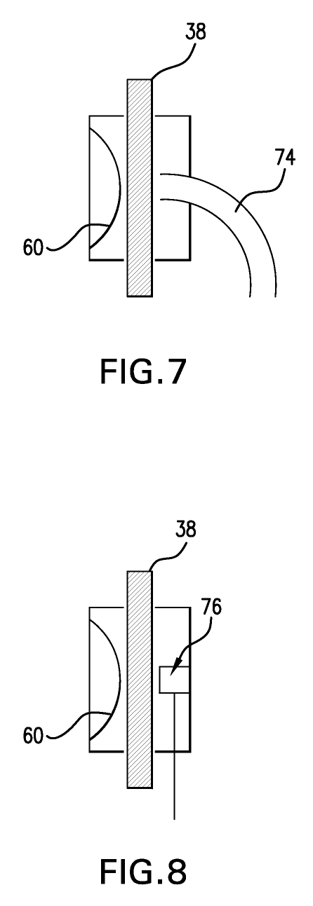

As shown in FIG. 7, an alternative embodiment can be provided that does not require the use of a roller to track the movement of the fiber 38 but instead uses direct fiber movement data as recorded by an optical fiber 74 optic transmission of observed fiber motion to a CMOS unit 76 (Complementary Metal Oxide Semi-Conductor Sensor) which when analyzed in the processor 14 yields movement data for the fiber 38.

Similar to the embodiment shown in FIG. 7, the alternative embodiment shown in FIG. 8 utilizes a CMOS unit 76; however, in this embodiment the CMOS is positioned directly adjacent to the moving fiber 38 and the recorded movement data is transmitted to the processor 14 through an electrically conductive wire.

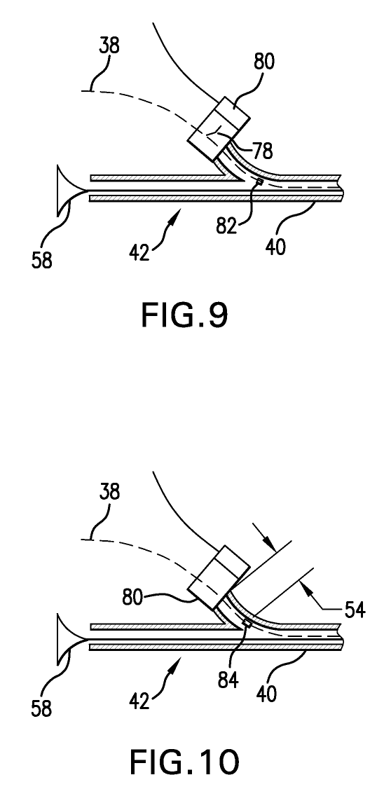

An alternative embodiment of the endoscope saving device 10, as shown in FIG. 9, includes a simple mechanical switch 78 within a tracking sensor 80 the mechanical switch 78 being physically moved by contact with a switch contact element 82 that is affixed on the fiber 38 at a predetermined position so as to initiate standby mode. While this embodiment in its most simple form would not also provide a warning tone for the user, it is within the concept of the invention that a second mechanical switch with a second sequentially aligned tracking sensor and switch contact element combination could be employed such that as the first combination effected a signal transfer to the processor 14, a warning tone could be emitted and as movement of the fiber 38 continued, the second combination of switch, sensor, and contact element would send a second signal to the processor that could initiate a standby mode for the device 10.

The alternative embodiment of the endoscope saving device 10 as shown in FIG. 10 can accomplish tracking of the fiber movement through the use of a magnetic/capacitive tracking unit 84 that is affixed to a pre-selected position on the fiber distal to a tracking sensor 80. As the fiber moves so does the affixed magnetic tracking unit 84, such that, when the fiber moves to a measured distance position near the tracking sensor, a signal is generated from the tracking sensor that is sent to the data processor by an electrically conductive wire.

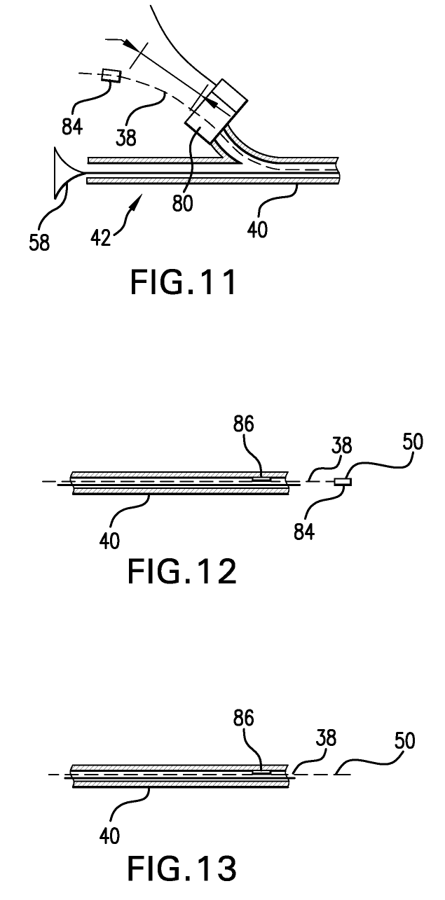

As shown in FIG. 11 an alternative embodiment of the endoscopic saving device 10 is provided wherein tracking of the fiber movement is accomplished as a magnetic/capacitive tracking unit 84 that is affixed to a pre-selected position on the fiber proximal to a tracking sensor, such as in the handle control of the laser. As the fiber 38 moves with the magnetic unit 84 to a measured distance position near the tracking sensor 80, a signal is generated by the tracking sensor 80 and transmitted to the data processor by an electrically conductive wire.

The alternative embodiment of the invention as shown in FIG. 12 accomplishes tracking of movement of the fiber 38 when a magnetic/capacitive tracking unit 84 that is affixed onto the distal tip of the fiber is drawn sufficiently close to a proximity sensor 86 located at the distal end of the endoscope structure such that a signal indicating close proximity of the magnetic tracking unit 84 to the proximity sensor 86 is sent by an electrically conductive wire to the data processor 14.

As shown in FIG. 13 another alternative embodiment of the present invention can be provided wherein tracking of the fiber movement is accomplished when the heat generating distal end of the fiber 50 comes into proximity to a heat sensor 88 affixed to the distal end of the endoscope structure 42 such that a signal is generated by the heat sensor 88 and transmitted to the data processor 14 by an electrically conductive wire.

While the device as described herein can be preferably used as a protective device component is an endoscope, it is within the inventors' understanding that the same principles of the invention can be adapted to conform to any endoscopic use of laser energy for medically directed diagnostic or surgical procedures.

The device 10 can be manufactured by methods known in the art or hereafter developed that are suitable of the making of medical devices, tools, or equipment. The components can be manufactured using materials having sufficient strength, resiliency, biocompatibility, and durability as is known to be required for such devices. By way of example only, suitable materials can include implant grade metallic materials, such as titanium, cobalt chromium alloys, stainless steel, or other suitable materials for this purpose. It is also conceivable that some components of the device can be made from plastics, composite materials, and the like.

The above described device can be used in a method for employing an endoscope that avoids damage from errant laser fire by providing the endoscope saving device 10 of the present invention and adapting that device to a selected endoscope, entering data relative to the selected endoscope into the data processor 14 of the device through the control interface 20, and operating the endoscope and laser unit so as to effect a diagnostic or surgical procedure wherein the endoscope is protected from errant laser fire.

Also provided is a kit, which includes at least one endoscope saving device provided in accordance with the present invention. The kit can also include additional devices and instruments; such as for example, diagnostic and surgical instruments capable of use with an endoscope and more particularly for use in endoscopic procedures. Such a kit can be provided with sterile packaging to facilitate opening and immediate use in an examining or operating room.

Each of the embodiments described above are provided for illustrative purposes only and it is within the concept of the present invention to include modifications and varying configurations without departing from the scope of the invention that is limited only by the claims included herewith.

What is claimed is:

1. A device for preventing damage to an endoscope from errant laser fire, the device comprising:
a tracking component being capable of connection to an endoscope having a laser fiber component, said tracking component being capable of collecting data on the distance said laser fiber extends past the distal end of said endoscope,
a data processor connected to said tracking component and capable of receiving and processing said collected data,
wherein said data processor is capable of determining from said processed data if said endoscope is in danger of receiving damage from errant laser fire and being capable of controlling said laser as required to prevent said damage.

2. The device of claim 1, wherein said device is configured to be capable of attaching to an endoscope at a fiber port seal of said endoscope.

3. The device of claim 1, wherein said device is configured to be capable of attaching to an endoscope in place of a fiber port seal of said endoscope.

4. The device of claim 1, wherein said device is configured to be capable of attaching to an inner sheath of an endoscope as a modified replacement for or attachment to a fiber port seal of said endoscope.

5. The device of claim 1, wherein said data processor is capable of calculating the distance said laser fiber extends past the distal end of said endoscope by analysis of said collected data in comparison to a stored data base of standard or custom endoscope lengths.

6. The device of claim 5, wherein said data processor is programmed to transmit a warning signal when said laser fiber is withdrawn to a potentially damaging proximity into said endoscope.

7. The device of claim 5, wherein said data processor is programmed to transmit a signal to put the laser in a standby mode when said laser fiber is withdrawn into said endoscope.

8. The device of claim 1, wherein said data processor can be programmed to include a database of standard or custom scope lengths for endoscopes to which the device can be attached.

9. The device of claim 1, wherein said data processor can be programmed to identify a specific type of endoscope to which said device can be connected.

10. The device of claim 8, wherein said data processor can analyze data provided by said tracking component to monitor the amount of laser fiber inserted into said endoscope and compare said amount of inserted laser fiber to said stored database.

11. The device of claim 1, wherein said tracking component comprises a roller unit component, said roller unit component being constructed to be capable of surviving chemical or heat sterilization processes.

12. The device of claim 11, wherein said roller unit component comprises at least one roller that is biased so as to maintain contact with said laser fiber, said at least one roller being capable of rotational movement corresponding to movement of said laser fiber into said endoscope,
whereas movement of said at least one roller is transmitted as data to said data processor.

13. The device of claim 12 wherein said roller unit component comprises two parallel aligned rollers each having contact with said laser fiber disposed between said two rollers.

14. The device of claim 1 further comprising a control interface capable of receiving data input for said data processor from a user.

15. The device of claim 12, wherein said at least one roller comprises reflective markings and said tracking component comprises an optical sensor capable of collecting data from relative movement of said reflective markings as said at least one roller rotates in correspondence with movement of said laser fiber.

16. The device of claim 1, wherein said data is transmitted from said tracking component to said data processor by an electrical connection.

17. the device of claim 1, wherein said data is transmitted from said tracking component to said data processor by an optical fiber connection.

18. The device of claim 1, wherein said device can be connected to an endoscope having a laser fiber wherein said laser fiber is provided with indicia and said tracking component of said device is capable of detecting relative movement of said indicia corresponding to movement of said laser fiber.

19. The device of claim 18, wherein said graduated indicia are analog encoded on said laser fiber.

20. The device of claim 18, wherein said graduated indicia are digitally encoded on said laser fiber.

21. The device of claim 18, wherein said indicia comprises a two stage label having a first stage label and a second stage label, said first stage label being capable of prompting said tracking device to transmit a first signal to said data processor and said second stage label being capable of prompting said tracking device to transmit a second signal to said data processor.

22. The device of claim 1, wherein said tracking component comprises an optical fiber, said optical fiber capable of receiving and transmitting data relating to movement of said laser fiber to said data processor.

23. The device of claim 1, wherein said tracking component comprises at least one mechanical switch capable of being moved by contact with a corresponding at least one contact point located on said laser fiber, movement of said at least one mechanical switch being capable of prompting said tracking component to transmit data to said data processor.

24. The device of claim 23, wherein said tracking component comprises a first switch and a second switch and said laser fiber comprises a first contact point and a second contact point, wherein movement of said first switch prompts transmission of data to the data processor for a warning signal output and movement of said second switch prompts transmission of data to the data processor to set the endoscope into standby mode.

25. The device of claim 1, wherein said tracking component is capable of sensing a magnetic field, said magnetic field being generated by a magnetic unit attached to said laser fiber at a position such that when said magnetic unit is close to said tracking unit, said magnetic sensor is prompted to transmit data to said data processor.

26. The device of claim 25, wherein said magnetic unit and magnetic sensor are located in a laser control handle.

27. The device of claim 25, wherein said magnetic unit is located proximate to a distal end of said laser fiber and said magnetic sensor is located at a distal end of said endoscope.

28. The device of claim 1, wherein said tracking component comprises a heat sensor located at a distal end of said endoscope, said heat sensor being capable of generating a signal to said data processor when heat from a distal end of said laser fiber is proximal to said end of said endoscope.

29. An endoscope comprises the device of claim 11.

30. A method of providing protection for an endoscope from errant laser fire, the method comprising;
providing an endoscope capable of being adapted to receive the device of claim 1;
attaching the device of claim 1 to said endoscope.

31. A kit comprising:
at least one of the devices of claim 1 and at least other instrument or tool capable for use to connect said device to an endoscope.
